(19) United States
(12) Patent Application Publication
(10) Pub. No.: US 2008/0274086 A1
(43) Pub. Date: Nov. 6, 2008

(54) Use of Cxcr4 Protein Expression on the Surface of Stem Cells as a Marker for Tumor Tropic Potential

(75) Inventors: John S. Yu, Los Angeles, CA (US); Moneeb Ehtesham, Nashville, TN (US)

Correspondence Address: DAVIS WRIGHT TREMAINE LLP/Los Angeles, 865 FIGUEROA STREET, SUITE 2400, LOS ANGELES, CA, 90017-2566, US

(73) Assignee: CEDARS-SINAI MEDICAL CENTER, Los Angeles, CA (US)

(21) Appl. No.: 10/598,468

(22) PCT Filed: Sep. 17, 2004
(86) PCT No.: PCT/US04/30607

§ 371 Date: Aug. 31, 2006

Related U.S. Application Data
(60) Provisional application No. 60/509,105, filed on Oct. 6, 2003.

(51) Int. Cl. A61K 35/12; C12N 5/08; A61P 35/00; C12Q 1/68

(52) U.S. Cl. 424/93.7; 435/368; 435/6

(57) ABSTRACT

The present invention relates to tumor tropic stem cells, and particularly to neural stem cells, and their use as delivery vehicles for therapeutic gene products to neoplastic foci. The stem cells with tumor tropic potential are selected based on the stem cells exhibiting CXCR4 receptors or an affinity for the chemokine SDF-1. The stem cells may additionally exhibit markers characteristic of astrocytic progenitors. The stem cells may be administered as part of a treatment regimen including the chemokine SDF-1.

### GOVERNMENT RIGHTS

The invention described herein arose at least in part in the course of or under Grant No. NS02232 awarded by the National Institutes of Health to Cedars-Sinai Medical Center. The government may have certain rights in this invention.

### FIELD OF THE INVENTION

This invention relates to treating and preventing various disease conditions, such as cancer.

### BACKGROUND OF THE INVENTION

Cancers of the central nervous system (CNS), also known as glial neoplasms, continue to be a research priority. Glial neoplasms include many heterogeneous tumors, such as astrocytomas, ependymal tumors, glioblastoma multiforme, and primitive neuroectodermal tumors. Although the incidence of malignant gliomas is low in comparison to other forms of cancer, glial neoplasms are both deadly and difficult to treat. In addition, despite advances in surgical techniques and adjuvant therapies, the prognosis for patients with malignant glial tumors remains dismal. For example, the most common and aggressive form of malignant glioma, glioblastoma multiforme, has a median survival time following diagnosis of under 1 year and a 2-year survival rate approaching zero (Surawicz, T. S. et al., “Brain tumor survival: results from the National Cancer Data Base,” J. Neurooncol., Vol. 40, p. 151-160 (1998)).

The failure of currently employed therapeutic approaches, which center on surgical resection followed by radiation and/or chemotherapy, is rooted in the highly disseminated nature of these tumors. High grade gliomas are highly infiltrative neoplasms, with solitary tumor cells or clusters of neoplastic cells migrating throughout the brain, often to significant distance from the main tumor. Despite aggressive therapy, it is almost impossible to successfully eliminate all of these tumor foci, which eventually serve as reservoirs for near universal tumor recurrence; thereby contributing to the inevitable lethality of this disease.

Standard adjuvant treatments including radiation and chemotherapy, despite having modest effects on long-term survival, have been unable to effect any meaningful impact on patient prognosis. The development of a successful therapeutic modality for malignant glioma will, therefore, center on the ability to devise a means of eliminating all viable intracranial neoplastic reservoirs left behind after surgical resection of the primary tumor mass. At present, this remains a daunting task given the highly disseminated nature of the disease process, and the current inability to adequately visualize and therapeutically target every remaining tumor cell.

One promising means of specifically directing treatment to migrating tumor satellites involves the use of neural stem cells (NSCs). NSCs are multipotent progenitor cells or neuronal glial precursors of the central nervous system that can be derived from embryonic, fetal, neonatal, or adult tissues and are capable of long-term, sustained in vitro propagation and terminal differentiation into a neuronal or glial fate (Cai, J. et al., “Properties of a fetal multipotent neural stem cell (NEP cell),” Dev. Biol., Vol. 251, p. 221-240 (2002)). Moreover, NSCs exhibit potent tropism for disseminating glioma cells in vivo, when inoculated into established intracranial gliomas in rodents. Specifically, NSCs migrate away from the primary site of injection and intersperse themselves with, or track into proximity of, tumor satellites that have spread away from the primary tumor mass making them a prime candidate for drug/treatment delivery (Aboody, K. S. et al., “Neural stem cells display extensive tropism for pathology in adult brain: evidence from intracranial gliomas,” Proc. Natl. Acad. Sci. USA, Vol. 97, No. 23 p. 12846-12851 (2000); Ehtesham, M. et al., “The use of interleukin 12-secreting neural stem cells for the treatment of intracranial glioma,” Cancer Res., Vol. 62, p. 5657-5663 (2002)). Further, treatment of cancer using other types of stem cells has also demonstrated success. For example, hematopoietic stem cells have been used to set up therapeutic strategies for the treatment of gynecological solid tumors such as ovarian cancer. (Perillo, A. et al., “Stem cells in gynecology and obstetrics,” Panminerva Med., Vol. 46, No. 1, p. 49-59 (2004)).

Stem cells engineered to secrete tumor toxic chemokines can, in this manner, deliver these therapeutic proteins directly to these disseminated neoplastic foci with significant bioactivity. In particular NSC populations secreting the immunostimulatory cytokines interleukin (IL)-12 and IL-4 as well as the pro-apoptotic protein tumor necrosis factor-related apoptosis-inducing ligand (TRAIL) have been used to target migrating tumor pockets with resulting decreases in tumor burden and prolongation in survival (Ehtesham, M. et al., “The use of interleukin 12-secreting neural stem cells for the treatment of intracranial glioma,” Cancer Res., Vol. 62, p. 5657-5663 (2002); Benedetti, S. et al., “Gene therapy of experimental brain tumors using neural progenitor cells,” Nat. Med., Vol. 6, No. 4 p. 447-450 (2000); Ehtesham, M. et al., “Induction of glioblastoma apoptosis using neural stem cell-mediated delivery of tumor necrosis factor-related apoptosis-inducing ligand,” Cancer Research, Vol. 62, p. 7170-7174 (2002)). Furthermore, the use of stem cells as therapeutic delivery vehicles has offered encouraging pre-clinical results.

However, the use of this technology in patients is still hampered by significant limitations, key among which is the isolation of clinically viable and legally utilizable sources of tumor tropic neural progenitors. Progress is, however, being made on this front as exemplified by recent reports regarding alternative tissue sources from which multipotent neural precursors can be derived (Jiang, Y. et al., “Pluripotency of mesenchymal stem cells derived from adult marrow,” Nature, Vol. 418, p. 41-49 (2002); Kabos, P. et al., “Generation of neural progenitor cells from whole adult bone marrow,” Exp. Neurol., Vol. 178, p. 288-293 (2002)).

Additional problems lie in the fact that the exact mechanisms governing the tropic behavior of stem cells are poorly understood. Early observations demonstrate that while many intratumorally inoculated stem cells exhibit robust migratory activity and tumor tracking capabilities, a significant proportion of transplanted stem cells do not exhibit this behavior and remained localized to the site of initial intracranial injection (Ehtesham, M. et al., “The use of interleukin 12-secreting neural stem cells for the treatment of intracranial glioma,” Cancer Res., Vol. 62, p. 5657-5663 (2002)). Given the abysmal prognoses associated with high grade gliomas, there is an urgent need to develop novel therapies with translational potential. Thus, there exists a need in the art for a method of treating and preventing infiltrative neoplasms.

### SUMMARY OF THE INVENTION

Described herein is an isolated stem cell useful for treating disease conditions in a mammal. This stem cell exhibits the CXCR4 receptor, markers characteristic of astrocytic differentiated stem cells and/or an affinity for the chemokine SDF-1, and may be administered to a mammal by any conventional means, such as, by way of example, intratumoral inoculation. The stem cell may be a neural stem cell (NSC). Furthermore, the stem cell of the present invention may be engineered to secrete cytotoxic cytokines for the treatment of disease conditions. Compositions including the stem cells of the present invention may further include an additional component, such as an adjuvant, to provide a therapeutically convenient formulation and/or to enhance biochemical delivery and efficacy of the stem cell. Furthermore, methods of treating or preventing cancer with the stem cells of the present invention are provided. Still further, methods of treating or preventing cancer with the stem cells of the present invention may optionally include concurrent treatment with the chemokine SDF-1.

Embodiments of the present invention provide methods for selecting stem cells with tumor tropic potential. The methods of the present invention include selecting stem cells based on the stem cells exhibiting the CXCR4 receptor and/or an affinity for the chemokine SDF-1. Further, the methods for selecting stem cells with tumor tropic potential in accordance with various embodiments of the present invention may further include selecting based on the presence of an additional marker, such as a marker characteristic of an astrocytic precursor, for example, A2B5 or glial fibrillary acidic protein (GFAP).

Embodiments of the present invention additionally provide methods of treating disease conditions in a mammal by use of the stem cells of the invention. The methods of the present invention include administering the stem cells by any conventional means, for example, intratumoral inoculation. Further, the methods of the present invention may include the administration of stem cells exhibiting CXCR4 receptors, an affinity for the chemokine SDF-1, and optionally, markers characteristic of astrocytic differentiated stem cells. The stem cells may be administered with an additional component such as an adjuvant, to provide a therapeutically convenient formulation and/or to enhance biochemical delivery and for efficacy of the composition. The methods of the present invention may include the administration of the chemokine SDF-1. Still further, the methods of the present invention may be useful in the treatment of various disease conditions, such as cancer.

Further embodiments of the present invention provide a kit for use in treating a mammal with the stem cells of the present invention. The kit of the present invention includes a volume of the stem cells of the invention along with instructions for their use in a manner consistent with the methods of the present invention. Further, the kit of the present invention may include a volume of the chemokine SDF-1.

### BRIEF DESCRIPTION OF THE DRAWINGS

The file of this patent contains at least one drawing executed in color. Copies of this patent with color drawing(s) will be provided by the Patent and Trademark Office upon request and payment of the necessary fee.

FIG. 1, which is executed in color, depicts NSC tropism for disseminating glioma in vivo in accordance with various embodiments of the present invention. NSCs-LacZ were inoculated into established intracranial GL26 tumors in C57B1/6 mice. Histological brain sections were then processed with routine X-gal staining, resulting in the development of a blue to dark blue precipitate within NSC-LacZ. Sections were then counterstained with neutral red. Tumor tissue could be identified by intense red staining of neoplastic nuclei and visible dense aggregates of tumor cells. T designates tumor, and N represents normal tissue. FIG. 1A is a low-power image illustrating the presence of nonmigratory NSC-LacZ within the main tumor mass (T), demarcated by arrows. FIG. 1B illustrates NSC-LacZ that have moved out of the main tumor mass and are moving into the proximity of tumor cell islets that are migrating along the grey matter/white matter boundary, likely along a white matter tract (inset box). Note that migratory NSC-LacZ are still aggregated in neurosphere-like accumulations. FIG. 1C represents a high-power magnification of the inset box in FIG. 1B. Dark blue NSC-LacZ aggregates are clearly visible in close proximity to a disseminating tumor satellite (T). FIG. 1D is a high-power magnification of an independent tumor satellite (demarcated by arrowheads) at significant distance from the primary tumor site. Blue NSC-LacZ are visible within the tumor, indicating that NSC-LacZ are capable of extensive migratory activity in vivo and can intercalate themselves into disseminated tumor islets.

FIG. 2, which is executed in color, depicts the results of histochemically analyzed brain tissue from glioma bearing animals that had received intratumoral inoculations of β-galactosidase expressing NSCs (NSCs-LacZ) in accordance with various embodiments of the present invention. NSCs-LacZ tracking disseminated glioma were subjected to routine X-gal staining, which revealed that a significant proportion of inoculated NSCs migrated away from the site of inoculation. Mirrored sections of those stains were then subjected to immunofluorescent histochemistry with a panel of antibodies specific for markers reflective of proteins expressed at varying stages of NSC differentiation. FIG. 2A shows a positive correlation between GFAP markers being indicative of tumor tropic NSCs-LacZ inoculated intratumorally. FIG. 2B shows a positive correlation between A2B5 markers and tumor tropic NSCs-LacZ in a tumor microsatellite. FIG. 2C shows a positive correlation between CXCR4 markers and tumor tropic NSCs-LacZ inoculated intratumorally. The A2B5 and GFAP markers are indicative of NSCs that have initiated differentiation pathways towards astrocytic and astroglial lineages. All images represent 400 times magnification.

FIG. 3 is a graphical representation of NSC migratory tropism towards glioma conditioned media in vitro in accordance with various embodiments of the present invention. Human and murine fetal NSCs were placed in the upper well of a two-well chemotaxis chamber system, separated from a lower well containing various media/culture supernatants by a polycarbonate membrane with multiple 5 micron pores. Following incubation at 37° C. for 4 hours, media from the lower chambers was harvested and cells quantified. Y-axis depicts percentage of NSCs that migrated into the lower chambers. FIG. 3A indicates that human fetal NSCs demonstrated minimal migratory activity towards normal unconditioned medium, whereas movement towards U87MG glioma supernatant was significantly higher (P=0.005; t-test). Dilution of glioma media resulted in a significant decrease in NSC chemotaxis (not shown) indicating that NSC translocation was likely due to a tumor elaborated soluble factor. Addition of a neutralizing antibody against one such potential factor, stromal-cell derived factor (SDF)-1, reduced chemotaxis noticeably compared to NSCs treated with isotype IgG, albeit not to a statistically significant extent (P=0.09; t-test). FIG. 3B indicates that murine fetal NSCs demonstrated enhanced migratory activity towards GL26 conditioned medium compared to control media (P=0.0001; t-test). Addition of an anti-CXCR4 neutralization antibody significantly decreased NSC translocation towards glioma conditioned media compared to NSCs treated with isotype IgG (P=0.003; t-test).

### DETAILED DESCRIPTION OF THE INVENTION

### A. Definitions

Unless defined otherwise, technical and scientific terms used herein have the same meaning as commonly understood by one of ordinary skill in the art to which this invention belongs. Singleton et al., Dictionary of Microbiology and Molecular Biology 2nd ed., J. Wiley & Sons (New York, N.Y. 1994); March, Advanced Organic Chemistry Reactions, Mechanisms and Structure 4th ed., J. Wiley & Sons (New York, N.Y. 1992); and Sambrook and Russel, Molecular Cloning: A Laboratory Manual 3rd ed., Cold Spring Harbor Laboratory Press (Cold Spring Harbor, N.Y. 2001), provide one skilled in the art with a general guide to many of the terms used in the present application.

One skilled in the art will recognize many methods and materials similar or equivalent to those described herein, which could be used in the practice of the present invention. Indeed, the present invention is in no way limited to the methods and materials described. For purposes of the present invention, the following terms are defined below.

“Alleviating” specific cancers and/or their pathology includes degrading a tumor, for example, breaking down the structural integrity or connective tissue of a tumor, such that the tumor size is reduced when compared to the tumor size before treatment. “Alleviating” metastasis of cancer includes reducing the rate at which the cancer spreads to other organs.

“Beneficial results” may include, but are in no way limited to, lessening or alleviating the severity of the disease condition, preventing the disease condition from worsening, curing the disease condition and prolonging a patient's life or life expectancy. The disease conditions may relate to or may be modulated by the central nervous system.

“Cancer” and “cancerous” refer to or describe the physiological condition in mammals that is typically characterized by unregulated cell growth. Examples of cancer include, but are not limited to, breast cancer, colon cancer, lung cancer, prostate cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, head and neck cancer, and brain cancer; including, but not limited to, astrocytomas, ependymal tumors, glioblastoma multiforme, and primitive neuroectodermal tumors.

“Conditions” and “disease conditions,” as used herein may include, but are in no way limited to any form of cancer; in particular, astrocytomas, ependymal tumors, glioblastoma multiforme, and primitive neuroectodermal tumors.

“Curing” cancer includes degrading a tumor such that a tumor cannot be detected after treatment. The tumor may be reduced in size or become undetectable, for example, by atrophying from lack of blood supply or by being attacked or degraded by one or more components administered according to the invention.

“Cytokine” is a generic term for proteins released by one cell population which act on another cell as intercellular mediators. Examples of such cytokines are lymphokines, monokines, and traditional polypeptide hormones. Included among the cytokines are growth hormones such as human growth hormone, N-methionyl human growth hormone, and bovine growth hormone; parathyroid hormone; thyroxine; insulin; proinsulin; relaxin; prorelaxin; glycoprotein hormones such as follicle stimulating hormone (FSH), thyroid stimulating hormone (TSH), and luteinizing hormone (LH); hepatic growth factor; fibroblast growth factor; prolactin; placental lactogen; tumor necrosis factor-α and -β; mullerian-inhibiting substance; mouse gonadotropin-associated peptide; inhibin; activin; vascular endothelial growth factor (VEGF); integrin; thrombopoietin (TPO); nerve growth factors (NGFs) such as NGF-β; platelet-growth factor; transforming growth factors (TGFs) such as TGF-α and TGF-β; insulin-like growth factor-I and -II; erythropoietin (EPO); osteoinductive factors; interferons such as interferon-α, -β, and -γ; colony stimulating factors (CSFs) such as macrophage-CSF (M-CSF), granulocyte-macrophage-CSF (GM-CSF), and granulocyte-CSF (G-CSF); interleukins (ILs) such as IL-1, IL-1α, IL-2, IL-3, IL-4, IL-5, IL-6, IL-7, IL-8, IL-9, IL-1, IL-11, IL-12 and IL-13; a tumor necrosis factor such as TNF-α or TNF-β; and other polypeptide factors including LIF and kit ligand (KL). As used herein, the term cytokine includes proteins from natural sources or from recombinant cell culture and biologically active equivalents of the native sequence cytokines.

“Exhibits” or “exhibiting” refers, generally, to the presence or display of something outwardly. For example, the terms may refer to the presence or display of a cell-surface marker or a transmembrane marker.

“Isolated” as used herein encompasses a purified neural stem cell that is substantially free of other cellular material, or culture medium when produced by recombinant techniques, or substantially free of chemical precursors or other chemicals when chemically synthesized.

“Mammal” as used herein refers to any member of the class Mammalia, including, without limitation, humans and nonhuman primates such as chimpanzees and other apes and monkey species; farm animals such as cattle, sheep, pigs, goats and horses; domestic mammals such as dogs and cats; laboratory animals including rodents such as mice, rats and guinea pigs, and the like. The term does not denote a particular age or sex. Thus, adult and newborn subjects, as well as fetuses, whether male or female, are intended to be included within the scope of this term.

“Neural Stem Cell” and “Neural Progenitor,” or NSC, refer to multipotent undifferentiated cells with the capacity for extensive proliferation that gives rise to more cells as well as progeny that can terminally differentiate into both neurons and the supporting glial cells.

“Pathology” of cancer includes all phenomena that compromise the well-being of the patient. This includes, without limitation, abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, invasion of surrounding or distant tissues or organs, such as lymph nodes, etc.

“Stem Cells” refer to omnipotent undifferentiated cells, derived from any tissue, with the capacity for extensive proliferation that gives rise to more cells as well as progeny that can terminally differentiate any tissue, including, for example, neural stem cells.

“Treatment” and “treating,” as used herein refer to both therapeutic treatment and prophylactic or preventative measures, wherein the object is to prevent or slow down (lessen) the targeted pathologic condition or disorder even if the treatment is ultimately unsuccessful. Those in need of treatment include those already with the disorder as well as those prone to have the disorder or those in whom the disorder is to be prevented. In tumor (e.g., cancer) treatment, a therapeutic agent may directly decrease the pathology of tumor cells, or render the tumor cells more susceptible to treatment by other therapeutic agents, e.g., radiation and/or chemotherapy.

“Tumor,” as used herein refers to all neoplastic cell growth and proliferation, whether malignant or benign, and all pre-cancerous and cancerous cells and tissues.

### B. Detailed Description

The present invention is based on the surprising discovery that the tumor tropic component of stem cell populations utilized in therapeutic models of intracranial glioma includes astrocytic precursors expressing significant levels of CXC chemokine receptor 4 (CXCR4), a chemokine receptor that is believed to govern cellular migration and homing in a variety of cell types, including neuronal and glial precursors in the developing brain. It has recently been reported that the production by glioma cells of stromal-cell derived factor-1 (SDF-1), the only known ligand for CXCR4, correlated with histological grade, tumor cell survival and invasiveness (Rempel, S. A. et al. “Identification and localization of the cytokine SDF1 an its receptor, CXC chemokine receptor 4, to regions of necrosis and angiogenesis in human glioblastoma,” Clin. Cancer Res., Vol. 6, p. 102-111 (2000); Barbero, S. et al., “Stromal cell-derived factor 1 alpha stimulates human glioblastoma cell growth through the activation of both extracellular signal-regulated kinases 1/2 and Akt,” Cancer Res., Vol. 63, p. 1969-1974 (2003)). The data upon which the inventive methods are at least partially based delineate important characteristics of the specific cells within generalized stem cell populations that exhibit the therapeutically relevant behavior of “seek and destroy” tumor tropic migration. Those characteristics are described in Ehtesham, M. et al., “The Use of Interleukin 12-secreting Neural Stem Cells for the Treatment of Intracranial Glioma,” Cancer Res., Vol. 62, p. 5657-5663 (2002), and Ehtesham, M. et al., “Glioma Tropic Neural Stem Cells Consist of Astrocytic Precursors and Their Migratory Capacity Is Mediated by CXCR4,” Neoplasia, Vol. 6, No. 3, p. 287-293 (2004). Those publications are hereby incorporated by reference herein in their entirety. The use of these markers and further work on the characterization of these migratory sub-populations will allow for refining of stem cell sub-populations that are increasingly responsive to cues that govern tropism for disseminated tumor satellites in vivo, and therefore allow for optimization of the therapeutic potential of stem cells in this setting.

Inoculation with stem cells is characterized by tumor tropic activity as well as stem cells that stay localized to the point of inoculation. This is the result of differing phenotypic profiles within in vivo inoculated stem cell populations. In this context, the tumor tropic capacity observed within stem cell inoculae is exhibited by a specific sub-population of stem cells at a particular stage of differentiation. In vivo glioma tracking stem cells express phenotypic markers, such as chemokine receptors, which indicate responsiveness to known chemotactic cues related to stem cell migration within the developing brain. These tracking stem cells that exhibit chemokine receptors also specific for malignant gliomas may be particularly effective in the treatment of cancer and other conditions receptive to stem cells.

In one embodiment of the present invention, isolated stem cells directed at malignant gliomas include those stem cells that exhibit CXCR4 receptors. Further, isolated stem cells may further include those stem cells that exhibit an affinity for the chemokine SDF-1. Isolated NSC may be particularly useful in connection with these embodiments of the present invention. In another embodiment of the present invention, the isolated tumor tropic stem cells used in connection with the present invention may also exhibit markers characteristic of astrocytic or astroglial differentiated stem cells; those stem cells with further tumor tropic potential. Again, NSC may be particularly appropriate stem cells in connection with this embodiment of the present invention. The markers may include A2B5 and/or GFAP, but may also include, without limitation, Sox-2, stage-specific embryonic antigen (SSEA)-1, S-100, Hes-1, Notch-1,4′,6′-diamidino-2-phenylindole (DAPI), embryonic form of neural cell surface molecule (E-NCAM), excitatory amino acid transporter (EAAT)1, EAAT2, platelet-derived growth factor receptor-alpha PDGFRα, cyclic 2′,3′-nucleotide-3′-phosphodiesterase (CNPase), and β-III tubulin; other functionally related markers may additionally and/or alternatively be present, and numerous further markers may also be present, as will be readily appreciated by those of skill in the art.

Further, in another embodiment of the present invention, the isolated stem cells exhibiting a CXCR4 receptor and/or other markers characteristic of astrocytic differentiation may be selected based on the stem cells exhibiting these receptors and markers. Still further, the isolated stem cells may be selected based on the stem cells exhibiting an affinity for the chemokine SDF-1. The selection of these stem cells based on the presence of these receptors and markers or affinity for chemokines may be readily accomplished by conventional methods by one of skill in the art without undue experimentation. For example, the method of selection may involve fluorescence-activated cell sorting (FACS), affinity columns, affinity beads, or any method which selectively binds the specific cell surface molecules. Alternatively, the method may use the cell surface molecules which are not expressed by stem cells to selectively remove or kill the undesirable cells, and, in this way, enrich for the desirable cells. Alternatively, the method can include the use of magnetic beads which selectively bind the stem cells.

The isolated stem cells may be suitable for use as a single agent, in a combination therapy, or with an additional component not enumerated herein as would be readily recognized by one of skill in the art.

Differentiation occurs when stem cells are contacted with certain factors. For example, when stem cells are grown in the presence of fetal calf serum, or other morphogenic agents, they can be differentiated into these various cell types or less primitive stem cells. NSCs, for example, will differentiate into neuronal and glial cells including neurons, glia, oligodendrocytes and astrocytes.

Many differentiation agents are known to one of skill in the art which can differentiate stem cells into specific types of nerve cells or other types of progenitors. Therefore, it is envisioned that the stem cells isolated herein may be differentiated by any means known to one of skill in the art. Some examples of differentiation agents include, but are not limited to, interferon gamma, fetal calf serum, nerve growth factor, removal of epidermal growth factor (EGF), removal of basic fibroblast growth factor (bFGF), neurogenin, brain-derived neurotrophic factor (BDNF), thyroid hormone, bone morphogenetic proteins (BMPs), Leukemia inhibitory factor (LIF), sonic hedgehog (shh), glial cell line-derived neurotrophic factors (GDNFs), vascular endothelial growth factors (VEGFs), interleukins, interferons, stem cell factor (SCF), activins, inhibins, chemokines, retinoic acid and ciliary neutrotrophic factor (CNTF). Furthermore, stem cells may be differentiated permanently or temporarily. For example, a stem cell can be temporarily differentiated to express a marker in order to use that marker for identification, and then the differentiation agent may be removed and the marker may no longer be expressed. However, it is to be understood that within the context of differentiation, agents such as interferon gamma, though inducing the expression of different markers, may not be classified as classical differentiation agents.

It is also to be understood that any anti-differentiation agents known to one of skill in the art may be used, including but not limited to: transforming growth factor (TGF)-β, TGF-α, EGF, FGFs, and delta (notch ligand).

In another embodiment of the present invention, the isolated tumor tropic stem cells used in connection with the present invention may be modified to express a heterologous gene encoding, for example, cytotoxic polypeptides involved in the treatment of cancer. For example α-, β- or γ-interferon, cytokines including IL-12, IL-4 and tumor necrosis factor, apoptotic proteins including TRAIL, protein kinases, protein phosphates and cellular receptors for any of the above are included. The heterologous gene may also encode enzymes involved in amino acid biosynthesis or degradation, purine or pyrimidine biosynthesis or degradation, and the biosynthesis or degradation of neurotransmitters, such as dopamine, or protein involved in the regulation of such pathways, for example protein kinases and phosphates. The heterologous gene may also encode transcription factors or proteins involved in their regulation, membrane proteins or structural proteins.

In one embodiment, the heterologous gene encodes a polypeptide for therapeutic use, which is beneficial in alleviating, curing or treating disease conditions. For example, of the cytokines and proteins described above, IL-12 and IL-4 are interleukins that significantly increase intratumoral CD4+ and CD8+ T-cell infiltration, and apoptotic protein TRAIL is an agonistic human monoclonal antibody that specifically binds to the TRAIL receptor protein expressed on solid tumors and tumors of hematopoietic origin to kill by apoptosis, or programmed cell death. Heterologous genes encoding these molecules may be particularly beneficial when used in accordance with the present invention.

In another embodiment of the present invention, the isolated tumor tropic stem cells may be modified to express a chemotherapeutic agent involved in the treatment of cancer. A “chemotherapeutic agent” is a chemical compound useful in the treatment of cancer. Examples of chemotherapeutic agents include alkylating agents such as thiotepa and cyclosphosphamide (CYTOXAN available from Bristol-Meyers; New York, N.Y.); alkyl sulfonates such as busulfan, improsulfan and piposulfan; aziridines such as benzodopa, carboquone, meturedopa, and uredopa; ethylenimines and methylamelamines including altretamine, triethylenemelamine, trietylenephosphoramide, triethylenethiophosphaoramide and trimethylolomelamine; nitrogen mustards such as chlorambucil, chlornaphazine, cholophosphamide, estramustine, ifosfamide, mechlorethamine, mechlorethamine oxide hydrochloride, melphalan, novembichin, phenesterine, prednimustine, trofosfamide, uracil mustard; nitrosureas such as carmustine, chlorozotocin, fotemustine, lomustine, nimustine, ranimustine; antibiotics such as aclacinomysins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, 5-FU; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; elformithine; elliptinium acetate; etoglucid; gallium nitrate; hydroxyurea; lentinan; lonidamine; mitoguazone; mitoxantrone; mopidamol; nitracrine; pentostatin; phenamet; pirarubicin; podophyllinic acid; 2-ethylhydrazide; procarbazine; polysaccharide-K (PSK available from Kureha Chemical; Japan); razoxane; sizofiran; spirogermanium; tenuazonic acid; triaziquone; 2,2′,2″-trichlorotriethylamine; urethan; vindesine; dacarbazine; mannomustine; mitobronitol; mitolactol; pipobroman; gacytosine; arabinoside (Ara-C available from Upjohn GmbH; Heppenheim, Germany); cyclophosphamide; thiotepa; taxanes, e.g. paclitaxel (TAXOL available from Bristol-Myers Squibb Oncology; Princeton, N.J.) and docetaxel (TAXOTERE available from Rhone-Poulenc Rorer; Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone; vincristine; vinorelbine; navelbine; novantrone; teniposide; daunomycin; aminopterin; xeloda; ibandronate; CPT-11; topoisomerase inhibitor RFS 2000; difluoromethylornithine (DMFO); retinoic acid; esperamicins; capecitabine; and pharmaceutically acceptable salts, acids or derivatives of any of the above. Also included in this definition are anti-hormonal agents that act to regulate or inhibit hormone action on cells such as anti-estrogens including for example tamoxifen, raloxifene, aromatase inhibiting 4(5)-imidazoles, 4-hydroxytamoxifen, trioxifene, keoxifene, LY117018, onapristone, and toremifene (FARESTON available from Orion Corp.; Finland); and anti-androgens such as flutamide, nilutamide, bicalutamide, leuprolide, and goserelin; and pharmaceutically acceptable salts, acids or derivatives of any of the above.

Engineering stem cells to express either a heterologous gene separate from the stem cell genome or chemotherapeutic agent may be conducted in any number of ways as would be readily recognized by one of skill in the art. For example, one common method involves in vitro infection of stem cells with a replication deficient adenovirus packaging a heterologous gene of interest (Liu, Y. et al., “In Situ adenoviral interleukin 12 gene transfer confers potent and long-lasting cytotoxic immunity in glioma,” Cancer Gene Ther., Vol. 9, p. 9-15 (2002); Schaack, J. et al., “Efficient selection of recombinant adenoviruses by vectors that express β-galactosidase,” J. Virol., Vol. 69, p. 3920-3923 (1995)). Still other methods exist employing retrovirus and other routine infectious agents (Ehtesham, M. et al., “The use of interleukin 12-secreting neural stem cells for the treatment of intracranial glioma,” Cancer Res., Vol. 62, p. 5657-5663 (2002); Benedetti, S. et al., “Gene therapy of experimental brain tumors using neural progenitor cells,” Nat. Med., Vol. 6, p. 447-450 (2000); Ehtesham, M. et al., “Induction of glioblastoma apoptosis using neural stem cell-mediated delivery of tumor necrosis factor-related apoptosis-inducing ligand,” Cancer Res., Vol. 62, p. 7170-7174 (2002); Cai, J. et al., “Properties of a fetal multipotent neural stem cell (NEP cell),” Dev. Biol., Vol. 251, p. 221-240 (2002)). Each of the aforementioned references is incorporated by reference herein in its entirety.

In another embodiment of the present invention, there is a method of using the CXCR4 receptor, the affinity for the chemokine SDF-1 and/or the astrocytic markers for identification of stem cells or of a subpopulation of stem cells with tumor tropic potential. The method may include providing stem cells and implementing a selection process that incorporates standard immunohistochemistry protocols as would be readily recognized by one of skill in the art. The immunohistochemistry protocols may include, without limitation, primary antibodies, chemokine receptors and other functionally related markers.

For example, the method may involve fluorescence-activated cell sorting (FACS), affinity columns, affinity beads, or any method which selectively binds the specific cell surface molecules. Alternatively, the method may use the cell surface molecules which are not expressed by stem cells to selectively remove or kill the undesirable cells, and, in this way, enrich for the desirable cells. Alternatively, the method can include the use of magnetic beads which selectively bind the stem cells.

Further, in various embodiments, the stem cells of the present invention may be combined with one or more additional components including, without limitation, a vehicle, an additive, a pharmaceutical adjunct, a therapeutic compound, a carrier and agents useful in the treatment of cancer or other disease conditions, and combinations thereof. Once so combined, the stem cells may be suitable for administration to a mammal to treat a disease condition; although formulation with such an additional component is not required to be administered. Still further, in various embodiments, the stem cells of the present invention may be part of a treatment regimen including the chemokine SDF-1 and the treatment regimen may be suitable for administration to a mammal to treat a disease condition.

Further, in one embodiment, the chemokine SDF-1 may be suitable for administration locally. Local delivery of a protein, such as SDF-1, may be accomplished by conjugating the selected protein to biocompatible or biodegradable macromolecules, e.g. biopolymers, lipids, polysaccharides, proteins including albumin and immunoglobulines, which have a particular receptor specificity. In this way a protein can be transferred to a particular part of the human body which is subject to treatment with the particular protein. Alternatively, the local delivery mechanism may comprise a targeting agent associated with the carrier material, the targeting agent capable of binding to a specific site within the individual. The targeting agent may be a protein or an antibody, such as a receptor antibody, an antitumor antibody, or a white blood cell antibody. According to the invention, the SDF-1 may be administered by a catheter-based intravascular or percutaneous delivery system, coated stent, parenteral, or pulmonary delivery. Other systemic methods of administration may include oral, intravenous, intraperitoneal, intramuscular administration, dermal and transdermal diffusion, nasal and other mucosal routes. Local intravascular administration by means of a catheter is a common technique in medical practice. For example, catheters as double balloon, porous balloon, microporous balloon, stent in a balloon, hydrogel, dispatch and iontophoresis may be used as will be appreciated by one of skill in the art.

Still further, a variety of proteins can be used to prepare stent coatings, including, but not limited to gelatin, collagen, albumin, and the like. Application of coatings may be accomplished by solvents including, but not limited to water, glycerin, N,N-dimethylformamide (DMF), and dimethylsulfoxide (DMSO). In alternative embodiments of the invention, it may be desirable to incorporate one or more additives in the coatings. Examples include surfactants, water-soluble drugs, biological agents, antimicrobial agents, and the like. Surfactants can improve the spreading property of the protein solution of the substrate. Useful surfactants include cationic surfactants, such as alkyl quaternary ammonium salts; anionic surfactants, such as sodium dodecyl sulfate; and non-ionic surfactants, such as poly(oxyethylene sorbitan monooleate). If the substrate is a device which is inserted into a blood vessel, such as an intravascular stent, a catheter, or an angioplasty balloon, it may be desirable to have as an additive a thrombogenic agent such as heparin. Additives which are anti-microbial agents such as sodium benzoate, can prevent bacterial growth on or around the substrate.

In another embodiment of the present invention, a kit is included comprising stem cells that exhibit CXCR4 receptors and/or an affinity for the chemokine SDF-1 and instructions for their use, for example, in treating a disease condition. The exact nature of the components configured in the inventive kit depends on its intended purpose and on the particular methodology that is employed. For example, some embodiments of the kit are configured for the purpose of alleviating, curing or treating cancer in a subject. In one embodiment, the kit is configured particularly for the purpose of delivering therapeutic treatments to glial neoplasms in a human subject.

Instructions for use may be included with the kit. “Instructions for use” typically include a tangible expression describing the steps for inoculating a subject with stem cells and/or for using the same in a therapeutic system. Optionally, the kit also contains other useful components, such as diluents, buffers, pharmaceutically acceptable carriers, specimen containers, syringes, stents, catheters, pipetting or measuring tools, and the like.

The materials or components assembled in the kit can be provided to the practitioner stored in any convenient and suitable way that preserves their operability and utility. For example, the components can be in dissolved, dehydrated, or lyophilized form; they can be provided at room, refrigerated, or frozen temperatures.

The components are typically contained in suitable packaging material(s). As employed herein, the phrase “packaging material” refers to one or more physical structures used to house the contents of the kit. The packaging material is constructed by well known methods, preferably to provide a sterile, contaminant-free environment. The packaging materials employed in the kit are those customarily utilized in the field. As used herein, the term “package” refers to a suitable solid matrix or material such as glass, plastic, paper, foil, and the like, capable of holding the individual kit components. Thus, for example, a package can be a glass vial used to contain suitable quantities of stem cells. The packaging material generally has an external label which indicates the contents and/or purpose of the kit and/or its components.

The above disclosure generally describes the present invention, and all patents and patent applications, as well as publications, cited in this disclosure are expressly incorporated by reference herein. A more complete understanding can be obtained by reference to the following Examples, which are provided for purposes of illustration only and are not intended to limit the scope of the invention.

### EXAMPLES

The following examples are typical of the procedures that may be used to select tumor tropic stem cells for the treatment of glial neoplasms, and to evaluate the efficacy of tumor tropic stem cell therapy which may be used to treat patients in accordance with various embodiments of the present invention. Modifications of these examples will be readily apparent to those skilled in the art who seek to treat patients whose condition differs from those described herein.

### Example 1

### Cells and Culture Process

The human U87MG, murine GL26 glioma cell lines, NIH 3T3, and 293 human embryonic kidney cell lines were cultured in DM/F12 (available from Invitrogen; Carlsbad, Calif.) and Dulbecco's Modified Eagle's medium (DMEM)(available from Invitrogen; Carlsbad, Calif.), respectively supplemented with 10% fetal bovine serum (obtained from Gemini Bio-Products; Calabasas, Calif.), L-glutamine and 1% penicillin/streptomycin (available from Invitrogen). Conditioned media from U87MG, GL26, NIH 3T3, or 293 cultures was obtained from confluent 75 cm² culture flasks seeded 96 hours earlier with approximately similar numbers of cells. Cryopreserved human fetal NSCs were obtained from Cambrex (Walkersville, Md.) and murine NSCs were harvested from the frontoparietal regions of day 15 mouse fetuses as described in Ehtesham, M. et al., “The use of interleukin 12-secreting neural stem cells for the treatment of intracranial glioma,” Cancer Res., Vol. 62, p. 5657-5663 (2002). NSCs were cultured in DM/F12 media (obtained from Invitrogen) supplemented with B-27 growth factor (obtained from Invitrogen), 1% penicillin/streptomycin (obtained from Invitrogen; Carlsbad, Calif.), 20 to 30 ng/ml human or murine epidermal growth factor, 20 to 30 ng/ml human basic fibroblast growth factor (Peprotech; Rocky Hill, N.J.), and 2 mg/ml heparin (Sigma; St. Louis, Mo.). Murine NSCs were engineered to express β-galactosidase by means of in vitro infection, with the LacZ gene bearing replication-defective adenovirus as described in Ehtesham, M. et al., “The use of interleukin 12-secreting neural stem cells for the treatment of intracranial glioma,” Cancer Res., Vol. 62, p. 5657-5663 (2002).

### Example 2

### Establishment of in Vivo Glioma Model and NSC Inoculation

Six to eight week old C57B1/6 mice (obtained from Charles River Laboratories; Wilmington, Mass.), were anesthetized with intraperitoneal ketamine and xylazine and stereotactically inoculated with 5×10⁴ GL26 cells in 3 μl of 1.2% methylcellulose/MEM in the right corpus striatum as reported in Ehtesham, M. et al., “Treatment of intracranial glioma with in situ interferon-gamma and tumor necrosis factor-alpha gene transfer,” Cancer Gene Ther., Vol. 9, p. 925-934 (2002). At day 7 post-implantation, animals received intratumoral inoculations of 2×10⁵ NSC-LacZ in 5 μl of serum and virus-free media, injected directly into established tumor using the same burr hole and stereotactic coordinates.

### Example 3

### Immunohistochemical Analysis of Glioma Tropic NSC Phenotypes

Brains harvested from NSC-LacZ inoculated tumor bearing animals were frozen on dry ice, sectioned using a cryostat, mounted on slides, and air-dried. For histological visualization of LacZ-expressing NSCs, sections were stained with X-gal as per routine protocol and then counterstained with neutral red. Adjacent tissue sections were fixed in acetone. Staining was performed using standard immunohistochemistry protocols using primary antibodies against β-galactosidase, Sox-2, SSEA-1, A2B5, E-NCAM, β-III tubulin, glial fibrillary acidic protein (GFAP), CNPase, PDGFRα (obtained from Chemicon; Temecula, Calif.), CXCR4 (obtained from Torrey Pines Biolabs; San Diego, Calif.), EAAT1 and EAAT2 (obtained from Santa Cruz Biotech; Santa Cruz, Calif.). Secondary staining was performed using antibodies conjugated with the fluorophores FITC or Cy3 (obtained from Chemicon). Following staining, slides were mounted in aqueous mounting media (obtained from ICN Biochemicals; St. Louis, Mo.) and visualized under a fluorescence microscope.

### Example 4

### In Vitro Chemotaxis Experiments

All chemotaxis experiments were performed using a chemotaxis chamber system (obtained from Neuro Probe; Gaithersburg, Md.) consisting of pairs of culture wells separated by a 5 μm porous polycarbonate membrane. Lower wells were filled with either GL26 or U87MG conditioned media harvested as described above. Fresh DMEM supplemented with 10% FBS and 1% penicillin/streptomycin was used as the unconditioned media control. Following placement of the intervening porous membrane, approximately 1.5×10⁵ disaggregated human or murine NSC were added to the top chambers. The chamber system was incubated at 37° C. for 4 hours after which media from lower wells was collected and quantitatively analyzed for cell content using flow cytometry against a defined number of fluorescent beads (obtained from BD Pharmingen; San Diego, Calif.). This allowed for quantification of the percentage of cells added to each top chamber that had migrated to the bottom chamber. For neutralization assays, anti-SDF-1 (250 μg/l) (neutralizing both known α and β isoforms of the chemokine) and anti-CXCR4 (40 μg/ml) monoclonal antibodies (obtained from R&D Systems; Minneapolis, Minn.) were incubated with tumor conditioned media or NSC, respectively, for 30 minutes at room temperature prior to the assay. Control samples were incubated with identical concentrations of an isotype matched non-specific antibody (obtained from BD Pharmingen). All experiments were performed in triplicate.

### Example 5

### Analysis of NSC Differentiation Subtype

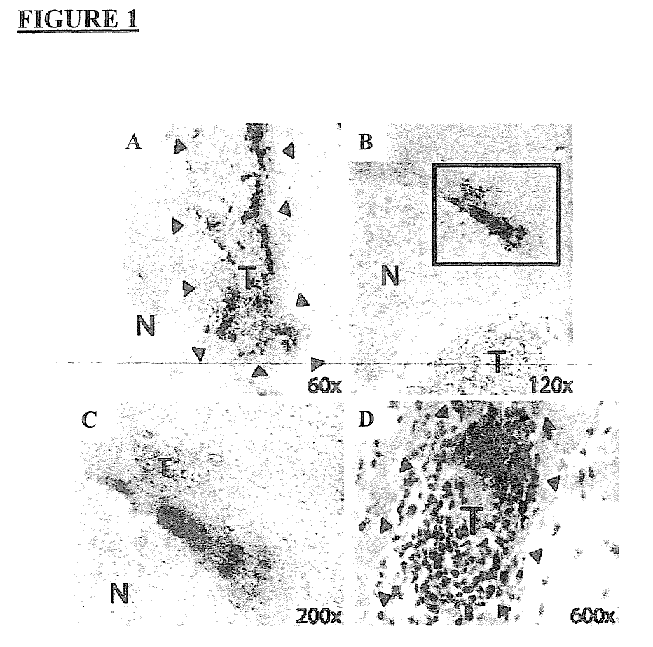

NSCs that migrate to sites of disseminating tumors include astrocytic precursors. Brain tissue from glioma bearing animals was histochemically analyzed after having received intratumoral inoculations of NSC-LacZ. Routine X-gal staining revealed a significant proportion of β-galactosidase positive cells that had migrated away from the site of inoculation into proximity of islets of tumor cells (readily identifiable following a neutral red counterstain) that were disseminating into and through normal brain parenchyma (FIG. 1), similar to findings reported previously (Ehtesham, M. et al., “The use of interleukin 12-secreting neural stem cells for the treatment of intracranial glioma,” Cancer Res., Vol. 62, p. 5657-5663 (2002)). At the same time, a residual population of NSC-LacZ remained localized to the site of initial inoculation and did not exhibit this migratory, tumor tropic activity. Mirror sections of the above mentioned samples (i.e., analogous histological samples that were not more than 20-30 μm removed from the original samples visualized with X-gal staining) were then subjected to immunofluorescent histochemistry with a panel of antibodies specific for markers reflective of proteins expressed at varying stages of NSC differentiation. These included the transcription factor Sox-2 and the cell surface stage-specific embryonic antigen-1 (SSEA-1), known to be expressed in uncommitted neural precursors; A2B5 and embryonic form of neural cell surface molecule (E-NCAM), indicative of NSC that have initiated differentiation pathways towards astrocytic and neuronal fates, respectively; GFAP, expressed in cells of astroglial lineages; excitatory amino acid transporter genes (EAAT1 and EAAT2), glutamate transporter related proteins found in functional, differentiated astroglial cells; platelet-derived growth factor receptor alpha (PDGFRα), expressed in oligodendroglial precursors; 2′,3′-cyclic nucleotide 3′-phosphodiesterase (CNPase), found in differentiated oligodendrocytes; and β-III tubulin, expressed in precursor as well as differentiated neuronal cells (Cai, J. et al., “Properties of a fetal multipotent neural stem cell (NEP cell),” Dev. Biol., Vol. 251, p. 221-240 (2002); Rao, M. S., “Multipotent and restricted precursors in the central nervous system,” Anat. Rec., Vol. 257, p. 137-148 (1999); Sutherland, M. L. et al., “Glutamate transporter mRNA expression in proliferative zones of the developing and adult murine CNS,” J. Neurosci., Vol. 16, p. 2191-2207 (1996); Cai, J. et al., “Identifying and tracking neural stem cells,” Blood Cells Mol. Dis., Vol. 31, p. 18-27 (2003); Capela, A. and S. Temple, “LeX/ssea-1 is expressed by adult mouse CNS stem cells, identifying them as nonependymal,” Neuron, Vol. 35, p. 865-875 (2002)). The focus was on the expression of these markers in NSC-LacZ that had dispersed from the primary inoculation tract and were now migrating in conjunction with or in proximity to disseminating tumor satellites, as observed on earlier X-gal stained mirror sections. The findings (summarized in Table 1) indicate that while populations of NSC expressing Sox-2 and SSEA-1 existed in the vicinity of the initial injection tract, the majority of β-galactosidase expressing NSC that were seen migrating along with glioma outgrowths and satellites were negative for these markers (not shown). Table 1 details the expression of protein markers associated NSCs-LacZ at varying stages of differentiation after in vivo intratumoral inoculation.

TABLE 1

| Differentiation | | Staining on non- | Staining on |
|---|---|---|---|
| stage related | Differentiation | migratory | glioma tropic |
| marker | stage | NSC-LacZ | NSC-LacZ |
| Sox-2 | Multipotent NSC | Weak, scattered | Negative |
| | | Cells | |
| SSEA-1 | Multipotent NSC | Weak, scattered | Negative |
| | | Cells | |
| A2B5 | Glial restricted | Positive | Positive |
| | precursor, astro- | | |
| | cyte restricted | | |
| | precursor, astrocyte | | |
| E-NCAM | Neuronal precursor, | Weak, scattered | Negative |
| | neuron | Cells | |
| PDGFRα | Oligodendroglial | Negative | Negative |
| | precursor, | | |
| | oligodendrocyte | | |
| GFAP | Astroglial precursor, | Strongly | Strongly |
| | astrocyte | Positive | Positive |
| B-III Tubulin | Neuron | Weak, scattered | Negative |
| | | Cells | |
| CNPase | Oligodendrocyte | Weak, scattered | Negative |
| | | Cells | |
| EAAT1/EAAT2 | Differentiated glia | Positive | Negative |
| | (primarily | | |
| | astrocytes) | | |

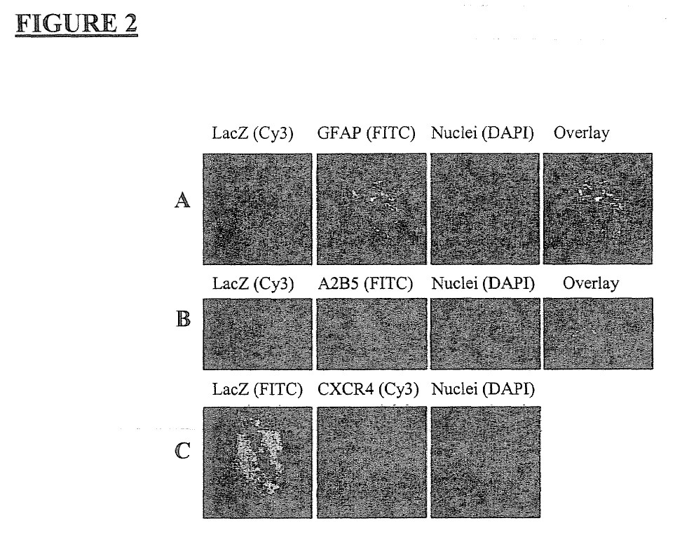

Additionally, these tumor tropic NSC populations were strongly positive for A2B5 and GFAP (FIG. 2), while negative for the oligodendroglial associated proteins PDGFRα and CNPase (not shown) as well as the neuronal marker β-III tubulin (not shown), clearly indicating differentiation towards astrocytic lineages. At the same time, these cells were negative for the glial specific glutamate transporter related proteins EAAT1 and EAAT2, known to be expressed in differentiated astrocytes (Sutherland, M. L. et al., “Glutamate transporter mRNA expression in proliferative zones of the developing and adult murine CNS,” J. Neurosci., Vol. 16, p. 2191-2207 (1996)). Conversely, populations of β-galactosidase positive cells with differentiated morphologies that expressed EAAT1 and EAAT2 along with GFAP and A2B5 could be observed in the vicinity of the initial injection tract within the main tumor mass (not shown), confirming that complete astrocytic differentiation of inoculated precursors was, in fact, taking place. However, the absence of EAAT1/EAAT2 expression in glioma tracking β-galactosidase positive cell populations, in conjunction with expression of A2B5 and clear absence of fully differentiated morphology, indicate that tumor tropic cell populations are comprised of progenitor cells that had initiated, but not completed, pathways towards astrocytic differentiation.

### Example 6

### Correlation Between Tumor Receptors and Glioma Tracking

Tumor tracking NSCs strongly express CXCR4. Based on the demonstrated ability of SDF-1 secretion from invasive glioma cells in promoting tumor invasiveness and survival (Barbero, S. et al., “Stromal cell-derived factor 1 alpha stimulates human glioblastoma cell growth through the activation of both extracellular signal-regulated kinases 1/2 and Akt,” Cancer Res., Vol. 63, p. 1969-1974 (2003); Zhou, Y. et al., “CXCR4 is a major chemokine receptor on glioma cells and mediates their survival,” J. Biol. Chem., Vol. 277, p. 49481-49487 (2002)), as well as the established role of this chemokine and its receptor CXCR4, in governing neuronal and glial precursor migration within the developing brain (Lazarini, F. et al., “Role of the alpha-chemokine stromal cell-derived factor (SDF-1) in the developing and mature central nervous system,” Glia, Vol. 42, p. 139-148 (2003); Reiss, K. et al., “Stromal cell-derived factor 1 is secreted by meningeal cells and acts as chemotactic factor on neuronal stem cells of the cerebellar external granular layer,” Neuroscience, Vol. 115, p. 295-305 (2002)), it was investigated whether tumor tracking NSC-LacZ populations expressed CXCR4. Weak CXCR4 expression was visible both on glioma cells as well as within NSC-LacZ populations remaining within the main tumor mass (not shown), whereas NSC-LacZ that were tracking tumor outgrowths and satellites strongly expressed this protein (FIG. 2), indicating a potential role for this receptor in governing NSC responsiveness to glioma elaborated chemotactic cues.

### Example 7

### NSC Migration Toward Tumor Conditioned Media in Vitro can be Inhibited by Blocking NSC Surface CXCR4 Receptors

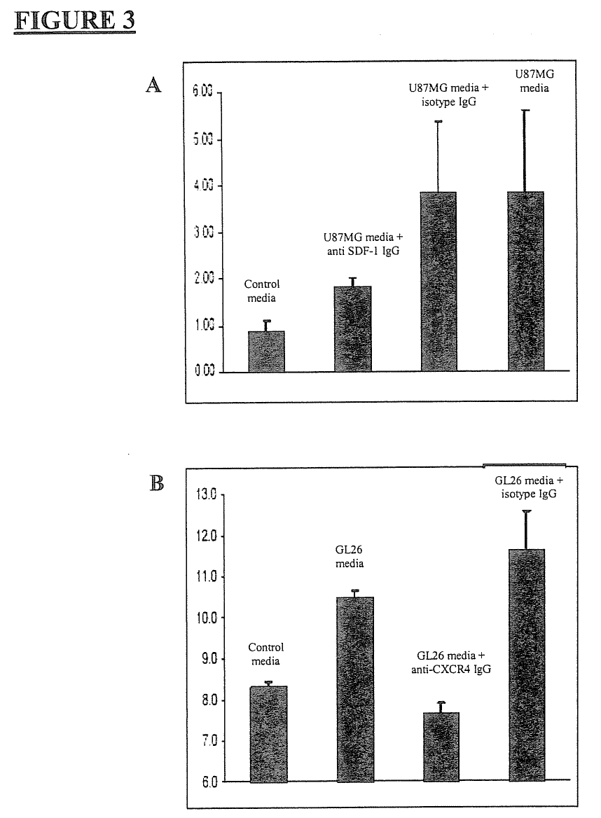

Based on the observation that tumor tropic NSC populations in vivo strongly expressed CXCR4, it was determined that this receptor played a role in NSC chemotaxis towards glioma. In a two-chamber based experimental system wherein tumor conditioned media was separated from human and murine NSC by a porous membrane, it was observed that NSC migration towards glioma supernatant was significantly higher than that observed towards normal media (FIG. 3), indicating chemotaxis towards a soluble factor present in tumor conditioned media. With the aim of determining whether neutralization of SDF-1 in tumor supernatant would inhibit NSC migration towards glioma conditioned media, anti-SDF-1 antibody was incubated with human U87MG glioma tumor supernatant and then utilized in a chemotaxis assay with human fetal NSC. It was found that in comparison to the significant NSC chemotaxis seen towards U87MG supernatant incubated with a non-specific IgG isotype antibody, addition of the anti-SDF-1 neutralization antibody markedly decreased NSC migration (FIG. 3A), although this difference did not meet statistical significance (P=0.09; t-test). This may represent a technical issue involving suboptimal neutralization of soluble chemokine versus more efficient blocking of cell surface CXCR4, or these findings may point to a role for additional, as of yet unidentified soluble ligand(s) for CXCR4, possibly further isoform variants of SDF-1 apart from the α and β subtypes we neutralized. However, following incubation with an anti-CXCR4 blocking antibody, a significant decrease in NSC migration towards glioma conditioned media was seen both in the case of murine (FIG. 3B) as well as human (not shown) fetal NSC (P=0.022 and P=0.003, respectively; t-test). In contrast, NSC incubated with an isotype matched non-specific antibody did not exhibit decreased migration towards tumor conditioned media when compared to untreated NSC (FIG. 3B). These data indicate that blocking of CXCR4 significantly inhibits NSC taxis towards glioma supernatant, suggesting an important role for this receptor in the tumor tropic behavior exhibited by these cells. The inability, however, to observe a statistically verifiable difference following neutralization of SDF-1 in tumor supernatants, may indicate either suboptimal neutralization of soluble chemokine or presence within the tumor conditioned media of secondary ligands capable of inducing chemotaxis through the CXCR4 pathway.

The level of NSC migration observed towards glioma conditioned media in vitro was significantly lower than that qualitatively predictable based on previously described in vivo migration patterns (Ehtesham, M. et al., “The use of interleukin 12-secreting neural stem cells for the treatment of intracranial glioma,” Cancer Res., Vol. 62, p. 5657-5663 (2002)). This is, however, in conjunction with the finding that tumor tropic behavior is exhibited principally by cells that are progressing towards astrocytic differentiation. As the cells utilized in the in vitro experiments comprised chiefly of NSCs cultured in conditions designed to favor maintenance of an undifferentiated state, although early evidence of eventual neuronal or glial directionality may still be discernable (Rao, M. S., “Multipotent and restricted precursors in the central nervous system,” Anat. Rec., Vol. 257, p. 137-148 (1999)), a lower percentage of committed and actively differentiating astrocytic precursors would be expected in these populations. Following in vivo transplantation, however, NSCs respond to predominantly gliogenic cues inherently present in the corpus striatum, increasing the numbers of astrocytic progenitors potentially responsive to chemotactic signals emanating from disseminating tumor cells.

Also of interest was the finding that primary murine fetal NSC exhibited significantly more migration, even towards unconditioned media, as opposed to human fetal NSCs. This may be explained by the differing origins of these cultures. Murine NSCs were derived from primary fetal tissue whereas human fetal NSCs were cultured from a several year old cryopreserved, commercially available stock. It is possible that freshly generated primary murine cells displayed a more active migratory capacity as opposed to the human NSCs, whose biological activity may have been hampered secondary to prolonged cryogenic storage.

While the description above refers to particular embodiments of the present invention, it will be understood that many modifications may be made without departing from the spirit thereof. The accompanying claims are intended to cover such modifications as would fall within the true scope and spirit of the present invention. The presently disclosed embodiments are therefore to be considered in all respects as illustrative and not restrictive, the scope of the invention being indicated by the appended claims, rather than the foregoing description, and all changes that come within the meaning and range of equivalency of the claims are therefore intended to be embraced therein.

What is claimed is:

1. An isolated stem cell, which is isolated by a method, comprising selecting the stem cell based on the stem cell exhibiting a CXCR4 receptor, demonstrating an affinity for the chemokine SDF-1, or both.

2. The isolated stem cell of claim 1, wherein said isolated stem cell exhibits markers characteristic of a precursor for astrocytic differentiated stem cells.

3. The isolated stem cell of claim 2, wherein said isolated stem cell exhibits an A2B5 astrocytic precursor marker.

4. The isolated stem cell of claim 2, wherein said isolated stem cell exhibits a glial fibrillary acidic protein (GFAP) astrocytic precursor marker.

5. The isolated stem cell of claim 1, wherein said isolated stem cell comprises a heterologous gene.

6. The isolated stem cell of claim 5, wherein said heterologous gene encodes a polypeptide of therapeutic use in the treatment of a disease condition.

7. The isolated stem cell of claim 6, wherein said polypeptide is cytotoxic.

8. The isolated stem cell of claim 6, wherein said polypeptide is involved in an immune response.

9. The isolated stem cell of claim 8, wherein said polypeptide is IL-12.

10. The isolated stem cell of claim 8, wherein said polypeptide is IL-4.

11. The isolated stem cell of claim 8, wherein said polypeptide is tumor necrosis factor-related apoptosis-inducing ligand (TRAIL).

12. The isolated stem cell of claim 1, wherein said isolated stem cell is a neural stem cell (NSC).

13. A method for assessing tumor tropic potential of a stem cell, comprising:
providing a stem cell;
determining an expression level of CXCR4 by the stem cell, an affinity by the stem cell for the chemokine SDF-1, or both; and
assessing tumor tropic potential of the stem cell based upon said expression level of CXCR4, said affinity for the chemokine SDF-1, or both.

14. The method of claim 13, wherein said stem cell exhibits markers characteristic of a precursor for astrocytic differentiated stem cells.

14. The method of claim 14, wherein said stem cell exhibits an A2B5 astrocytic precursor marker.

16. The method of claim 14, wherein said stem cell exhibits a glial fibrillary acidic protein (GFAP) astrocytic precursor marker.

17. The method of claim 14, wherein said stem cell is a neural stem cell (NSC).

18. A method for treating a disease condition in a mammal, comprising:
providing a stem cell that exhibits a CXCR4 receptor, that demonstrates an affinity for the chemokine SDF-1, or both; and
administering said stem cell to said mammal in an amount sufficient to treat said disease condition.

19. The method of claim 18, wherein said stem cell is an astrocytic progenitor cell.

20. The method of claim 18, wherein said stem cell exhibits markers characteristic of a precursor for astrocytic differentiated stem cells.

21. The method of claim 18, wherein said stem cell exhibits an A2B5 astrocytic precursor marker.

22. The method of claim 18, wherein said stem cell exhibits a glial fibrillary acidic protein (GFAP) astrocytic precursor marker.

23. The method of claim 18, wherein said stem cell comprises a heterologous gene.

24. The method of claim 23, wherein said heterologous gene encodes a polypeptide of therapeutic use in the treatment of said disease condition.

25. The method of claim 24, wherein said polypeptide is cytotoxic.

26. The method of claim 24, wherein said polypeptide is involved in an immune response.

27. The method of claim 26, wherein said polypeptide is IL-12.

28. The method of claim 26, wherein said polypeptide is IL-4.

29. The method of claim 26, wherein said polypeptide is tumor necrosis factor related apoptosis-inducing ligand (TRAIL).

30. The method of claim 18, wherein the disease condition is selected from the group consisting of breast cancer, colon cancer, lung cancer, prostate cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, head and neck cancer, astrocytomas, ependymal tumors, glioblastoma multiforme, and primitive neuroectodermal tumors.

31. The method of claim 18, wherein administering said stem cells further comprises administering said stem cells in a composition further comprising an additional component selected from the group consisting of a vehicle, an additive, a pharmaceutical adjunct, a therapeutic compound, a carrier, agents useful in the treatment of disease conditions, and combinations thereof.

32. The method of claim 18, further comprising administering a volume of the chemokine SDF-1 to said mammal.

33. The method of claim 18, wherein said stem cell is a neural stem cell (NSC).

34. A kit comprising:
a volume of stem cells that exhibit a CXCR4 receptor, the demonstrate an affinity for the chemokine SDF-1, or both; and
instructions for the use of said volume of stem cells in the treatment of a disease condition in a mammal.

35. The kit of claim 34, wherein said volume of stem cells are included in a composition that further comprises an additional component selected from the group consisting of a vehicle, an additive, a pharmaceutical adjunct, a therapeutic compound, a carrier, agents useful in the treatment of disease conditions, and combinations thereof.

36. The kit of claim 34, wherein the disease condition is selected from the group consisting of breast cancer, colon cancer, lung cancer, prostate cancer, hepatocellular cancer, gastric cancer, pancreatic cancer, cervical cancer, ovarian cancer, liver cancer, bladder cancer, cancer of the urinary tract, thyroid cancer, renal cancer, carcinoma, melanoma, head and neck cancer, astrocytomas, ependymal tumors, glioblastoma multiforme, and primitive neuroectodermal tumors.

37. The kit of claim 34, wherein said stem cell is a neural stem cell (NSC).

38. The kit of claim 34, further comprising a volume of the chemokine SDF-1, and instructions for the use of said volume of the chemokine SDF-1 in the treatment of the disease condition.
